(19) United States
(12) Patent Application Publication
(10) Pub. No.: US 2026/0061144 A1
(43) Pub. Date: Mar. 5, 2026

(54) INHALATION DEVICE AND METHOD

(71) Applicant: INVOX BELGIUM NV, Diepenbeek (BE)

(72) Inventors: Frank Bartels, Hattingen (DE); Jürgen RAWERT, Koeln (DE)

(21) Appl. No.: 19/318,438

(22) Filed: Sep. 4, 2025

Related U.S. Application Data
(63) Continuation of application No. 18/175,301, filed on Feb. 27, 2023, now Pat. No. 12,434,022.
Application No. 18/175,301 is a continuation of application No. 16/609,182, now Pat. No. 11,612,704.
(60) Provisional application No. 62/491,965, filed on Apr. 28, 2017.

(30) Foreign Application Priority Data
Apr. 28, 2017 (EP) 17168869.0

(51) Int. Cl. A61M 15/00; A61M 11/00

(52) U.S. Cl. CPC A61M 15/0086; A61M 11/007; A61M 15/0065; A61M 15/009; A61M 2202/0468; A61M 2205/3337; A61M 2205/3351; A61M 2205/3355; A61M 2205/8281

(57) ABSTRACT

An inhalation device for medically active liquids for generation of an aerosol comprises a housing, inside this housing a reservoir for storing a liquid, a pumping device with a pumping chamber for generation of a pressure inside said pumping chamber, wherein the pumping chamber is fluidically connected with the reservoir via a check valve which blocks in direction of the reservoir, a riser pipe which can be received with at least one reservoir-facing, interior end in said pumping chamber, and a nozzle which is connected liquid-tight to an exterior end of the riser pipe, wherein the interior volume of the pumping chamber is changeable by means of relative motion to the riser pipe, and wherein the riser pipe is immobile and firmly attached to the housing or to the nozzle, and the pumping chamber is moveable relative to the housing or to the nozzle.

### CROSS-REFERENCE TO RELATED APPLICATIONS

This application is a continuation of U.S. application Ser. No. 18/175,301, filed Feb. 27, 2023, which is a continuation of U.S. application Ser. No. 16/609,182, filed Oct. 28, 2019, which is a United States Application under 35 U.S.C. § 371 claiming priority to and the benefit of PCT Application No. PCT/EP2018/061056, filed Apr. 30, 2018, which claims priority to and the benefit of European Application No. 17168869.0, filed on Apr. 28, 2017, and U.S. Provisional Application Ser. No. 62/491,965, filed on Apr. 28, 2017, the contents of each of which are hereby incorporated by reference in their entireties.

### FIELD OF THE INVENTION

The invention relates to the field of inhalation devices for liquids. In particular, the invention relates to an inhalation device having improved flow characteristics, and to a method for the generation of an aerosol of a medically active liquid by means of an inhalation device.

### BACKGROUND OF THE INVENTION

Nebulizers or other aerosol generators for liquids are known from the art since a long time ago. Amongst others, such devices are used in medical science and therapy. There, they serve as inhalation devices for the application of active ingredients in the form of aerosols, i.e. small liquid droplets embedded in a gas. Such an inhalation device is known e.g. from document EP 0 627 230 B1. Essential components of this inhalation device are a reservoir in which the liquid that is to be aerosolized is contained; a pumping device for generation of a pressure being sufficiently high for nebulizing; as well as an atomizing device in the form of a nozzle. By means of the pumping device, the liquid is drawn in a discrete amount, i.e. not continuously, from the reservoir, and fed to the nozzle. The pumping device works without propellant and generates pressure mechanically.

A known embodiment of such an inhalation device is presented in document WO 91/14468 A1. In such a device, the pressure in the pumping chamber which is connected to the housing is generated by movement of a moveable hollow piston. The piston is moveably arranged inside the immobile pumping chamber. The (upstream arranged) inlet of the hollow piston is fluidically connected to the interior of the reservoir (reservoir pipe section). Its (downstream arranged) tip leads into the pumping chamber. Furthermore, a check valve that inhibits a back flow of liquid into the reservoir is arranged inside the tip of the piston.

For filling the piston, the same is directly connected with its upstream end to the reservoir. By pulling out the piston of the pumping chamber, its interior volume is enlarged, such that an increasing under pressure is built up inside the pumping chamber. This pressure propagates through the hollow piston into the reservoir, such that liquid is sucked from the same into the piston. At the same time, said valve opens at its tip, since the pressure inside the reservoir is higher than inside the (yet empty) pumping chamber. The pumping chamber is being filled. At the same time, a spring is loaded, and locked at the motion's end when the moveable piston has reached its lower dead center and the pumping chamber is filled.

The spring can be manually unlocked. The stored energy is then abruptly released. The piston is again pushed in direction of the pumping chamber and into the same, thus decreasing its interior volume. The aforementioned check valve is now closed, such that a growing pressure builds up inside the pumping chamber, since the liquid is inhibited from flowing back into the reservoir. Eventually, this pressure results in ejection of the liquid from the nozzle which is arranged at the downstream end of the pumping chamber.

In order to face the risk of a reverse flow of already ejected liquid or even outside air, a further check valve, subsequently being called outlet valve, can be arranged at the downstream end of the pumping chamber just before the nozzle, allowing emitted liquid to pass, but blocking incoming gas.

The piston is arranged inside the pressure spring which is designed as helical spring, thus limiting its outside diameter. Also because of the typically small volume (e.g. 15 μl), the piston is designed with a thin interior (and often also exterior) diameter.

This typically small inner diameter of the moveable piston (e.g. 0.3 to 1.0 mm), together with a small size of the check valve being arranged within, is a drawback of the described construction. The small diameter results in a high flow resistance, such that in particular, media of higher viscosities flow into and through the piston only very slowly. In other words, the described construction is suitable especially for low-viscosity (aqueous) liquids and for emitting low doses thereof. Furthermore, fabrication of a sufficiently tight check valve of small diameter is difficult.

The object of the invention is the provision of a device that avoids the drawbacks of the known art.

The inhalation device shall also allow for ejection of media of higher viscosities in a short time, and with high reproducibility.

### DESCRIPTION OF THE INVENTION

The object is solved by a device according to the disclosure, as well as a method according to the disclosure. Advantageous embodiments are described in the respective dependent claims, the subsequent description, as well as the accompanying figures.

The inhalation device according to the invention serves for the generation of an aerosol of medically active liquids, and in particular, of such aerosols which can be inhaled.

The inhalation device comprises a housing, which preferably can be held comfortably with one hand. Arranged inside this housing, and optionally connected or connectable with the same, is a reservoir for storing the medically active liquid, and a pumping device with a pumping chamber for generation of a pressure inside said pumping chamber, wherein the pumping chamber is fluidically connected with the reservoir, optionally by means of a reservoir pipe (or reservoir pipe section), via a check valve which blocks in direction of the reservoir. Thus, the check valve allows a liquid flow from the reservoir into the pumping chamber, and blocks a flow in opposite direction.

The inhalation device further comprises a riser pipe having at least one reservoir-facing, interior end which can be received in said pumping chamber, and a nozzle which is connected liquid-tight directly or indirectly to an exterior end of the riser pipe.

The interior volume of the pumping chamber is changeable by means of relative motion of the pumping chamber to the riser pipe in that the riser pipe increases the volume by being pushed into, and decreases the volume by being pulled out of the pumping chamber. The term “interior volume” describes the volume which extends from the reservoir-facing inlet of the pumping chamber to the place where the interior end of the riser pipe is located.

The inhalation device according to one embodiment of the invention is characterized in that the riser pipe is immobile and firmly, directly or indirectly, and/or permanently or detachably, attached to the housing, while the pumping chamber is moveable relative to the housing. In other words, the riser pipe maintains its position relative to the housing, and the pumping chamber can alter its position relative to the housing, and in particular, along a longitudinal axis of the same, such as to perform a piston-in-cylinder-type movement of the immobile riser pipe in the moveable pumping chamber.

In another embodiment, the immobility of the riser pipe is primarily related to the nozzle, rather than to the housing. Thus, nozzle and riser pipe form—in terms of movability—one unit. However, if the nozzle itself is immobile with respect to the housing, this is also true for the riser pipe, thus arriving at the firstly described embodiment.

An advantage of these solutions is that the passage between pumping chamber and reservoir can be designed with less restrictions compared to the known solution. It is e.g. possible to design a significantly larger check valve, which is easier to manufacture, since it does not have to be contained within the hollow piston known from the art. As a result, the size of the check valve is mainly only restricted by the interior size of the housing or, if such a construction is desired, the inner size of a spring that surrounds the pumping device. The (approximate) identity of the diameter of valve, riser pipe and reservoir pipe, as known from the art, becomes obsolete. Furthermore, since no movable piston needs to be connected to the reservoir, the component which enters the reservoir and the moveable component (i.e. the pumping chamber) can be designed independent of each other, allowing to better suit the individual functions. In this respect, the invention provides for higher design flexibility because the moveable pumping chamber, due to its robust structure and dimensions, provides better opportunities for designing a mechanically stable connection with the reservoir than does the moveable riser pipe which is typically less robust. Also, the connection between pumping chamber and reservoir can be designed with a larger diameter, such that higher flow velocities and fluid viscosities become feasible. Further, a support for the reservoir can be integrated into the component that comprises the pumping chamber. Additionally, the vent for pressure equilibration of the reservoir can be moved away from the reservoir body itself to, e.g., a connector which forms an interface between reservoir and pumping chamber, facilitation the construction and avoiding the necessity to provide an essentially “open” reservoir body.

According to a preferred embodiment, the nebulizer comprises further a means for the storage of potential energy, the device being coupled to the pumping chamber and being lockable in a loaded position, wherein upon unlocking, the stored energy is transformable into a motion of the pumping chamber.

Preferably, the means for the storage of potential energy is designed as tension or pressure spring. However, besides a metallic or plastic body, also a gaseous medium, or magnetic force utilizing material can be used as means for energy storage. By compressing or tensioning, potential energy is fed to the means. One end of the means is supported at or in the housing at a suitable location; thus, this end is essentially immobile. With the other end, it is connected to the pumping chamber; thus, this end is essentially moveable. The means can be locked after being loaded with a sufficient amount of energy, such that the energy can be stored until unlocking takes place. Thus, when being unlocked, the means can release the potential energy (e.g. spring energy) to the pumping chamber, which is then powered and moves. Typically, the energy release takes place abruptly, so that a high pressure can build up inside the pumping chamber.

The means for storing potential energy can also be provided in the form of a highly pressurized gas container. By suitable arrangement and repeatable intermittent activating (opening) of the same, part of the energy which is stored inside the gas container can be released to the pumping chamber. This process can be repeated until the remaining energy is insufficient for once again building up a desired pressure in the pumping chamber. Then, the gas container must be refilled or exchanged.

According to one embodiment, the check valve is adapted to open only when the pressure difference between the upstream and the downstream side of the valve, i.e. the reservoir and the pumping chamber side, is above a predefined threshold value, and remains closed as long as the pressure difference is below the threshold value. “Pressure difference” means that, irrespective of the concrete pressure values, only the relative pressure difference between the two sides is relevant for determining whether the check valve blocks or opens. If, for example, the pressure on the upstream (reservoir) side is already positive (e.g. due to thermal expansion), but the pressure on the downstream (pumping chamber) side is ambient pressure (no activation of the device), the pressure difference is below the threshold value, which allows the valve to stay closed even when subject to a positive pressure in opening direction. This means that the check valve remains closed until the threshold pressure is met, thus keeping the passage between reservoir and pumping chamber safely shut e.g. when the inhalation device is not in use. Examples for threshold pressure differences are in the range of 1-1000 mbar, and more preferably between 1 and 20 mbar.

Only upon activation of the pumping device, by building up a high pumping chamber pressure, the pressure difference (due to a high pressure in the pumping chamber, and a significantly lower pressure in the reservoir, resulting in a large pressure difference) becomes high enough and exceeds the threshold value of the pressure difference, so that the check valve finally opens and allows the pressure chamber being filled with liquid from the reservoir.

A simple device operating with such a threshold pressure difference is a ball valve pre-loaded with a spring. The spring pushes the ball into its seat, and only if the pressure acting against the spring force exceeds the latter, the ball valve opens.

Other valve types which—depending on their construction—may operate with such a threshold pressure difference are duckbill valves or flap valves.

The advantage of such a valve operating with a threshold pressure difference is that the reservoir can be kept closed until active use is being made of the inhalation device, thus reducing unwanted splashing of reservoir liquid during device transport, or evaporation during long-term storage of the device.

According to a further embodiment, an inhalation device comprises an outlet valve inside the riser pipe for avoiding a return flow of liquid or air into the exterior end of the same.

Typically, said end of the riser pipe is located close to the nozzle. The nozzle is in fluidic communication with the outside air. After emitting the amount of liquid which is collected inside the pumping chamber through the nozzle, along with pushing the pumping chamber onto the immobile riser pipe, the pumping chamber must be refilled. For this, the pumping chamber is largely pulled off from the riser pipe, so that the interior volume of the pumping chamber increases. Along with this, an underpressure is generated inside the pumping chamber which causes liquid to be sucked into the pumping chamber from the reservoir which is located upstream of the pumping chamber. However, said underpressure also propagates downstream through the riser pipe up to the outside of the nozzle and could lead to air being sucked into the device through the nozzle opening(s). This problem can be avoided by providing a check valve that opens towards the nozzle opening(s), and blocks in the opposite direction.

Such check valve may not always be necessary. It should be noted that the present construction generally allows for a relatively large design of the pumping chamber inlet, resulting in a quick inflow of liquid into the pumping chamber. At the same time, the channel(s) of the nozzle has or have typically a relatively small cross section, resulting in a slow back flow (with identical underpressure in the pumping chamber). With an accordingly large difference of the inlet and outlet channel cross-sections as a prerequisite, it is optionally possible to omit the described check valve.

According to another embodiment, the inhalation device comprises an outlet valve between riser pipe and nozzle for avoiding a return flow of liquid or air towards the exterior end of the riser pipe. According to this embodiment, the outlet valve is not arranged within the (typically very narrow) inside of the riser pipe, but downstream of the same, e.g. in a separate connector. The latter can provide a somewhat larger diameter, such that more space is available for the design of the outlet valve, thus simplifying the same.

Optionally, the outlet valve can be of a type that blocks below (and opens above) a threshold pressure difference as described above. If a spring is used, the spring force must be directed against the pumping chamber/reservoir such that only when the difference between the interior pressure of the pumping chamber and the ambient pressure exceeds the threshold pressure difference value, the outlet valve opens. The advantages of such a valve correspond to the respective aforementioned advantages.

According to one embodiment, the reservoir is firmly attached to the pumping chamber and thus moveable inside the housing. This means that in each ejection cycle, the reservoir moves together with the pumping chamber from the initial position, in which the pumping chamber has its maximum interior volume, to the end position, in which the same is minimal, and eventually back to the initial position. As used herein, the expression “firmly attached” includes both permanent and non-permanent (i.e. releasable) forms of attachment. One of the advantages of this construction is that it provides the smallest possible dead volume between reservoir and pumping chamber. According to another embodiment, the reservoir is connected to the pumping chamber by means of a flexible element such as e.g. a hose, and firmly attached to the housing. Thus, according to this embodiment, the reservoir does not move along with the pumping chamber, but is firmly (but, however, typically detachably) attached to the housing. One advantage of this construction is that the energy which is abruptly released upon unlocking acts solely onto the pumping chamber for accelerating the same, but not also onto the reservoir which typically—and in particular at the beginning of its usage—can have a relatively large mass. A higher acceleration of the pumping chamber, and thus, a higher pressure, is the result.

In one embodiment, the reservoir is designed to be collapsible, such as by means of a flexible or elastic wall. The effect of such design is that upon repeated use of the device which involves progressive emptying of the reservoir, the flexible or elastic wall buckles or folds such as to reduce the internal volume of the reservoir, so that the under pressure which is necessary for extraction of a certain amount of liquid does not increase substantially over use. A similar effect can be achieved when a rigid container has a moveable bottom by means of which the interior volume of the reservoir can also be successively be reduced.

According to another embodiment, the reservoir is designed as a collapsible bag. The advantage of a collapsible bag is that the inside pressure of the reservoir is almost independent of the filling level, and the influence of thermal expansion is almost negligible. Also, the construction of such a reservoir type is rather simple and already well established.

The invention also relates to a method for the generation of an aerosol of a medically active liquid by means of an inhalation device, and preferably, of the aforementioned type.

The main components of such a device are a housing, a pumping device with a pumping chamber, a riser pipe, and a nozzle. Of course, additional components such as check valves etc. are preferably part of the inhalation device as well. In order to avoid repetition, reference is made to the according sections above, as well as the figures and their description below.

The method comprises two main steps:
(I) moving an interior end of the riser pipe into the interior volume of the pumping chamber such that the latter is reduced and a pressure of a liquid contained within this volume is increased;
(II) ejecting said liquid (F) through an exterior end (5B) of said riser pipe (5) and the nozzle (6) such that a spray is formed.

According to the invention, during said motion, with regard to housing and/or nozzle, said riser pipe stays immobile, while said pumping chamber alters its relative position. In other words, the pumping chamber is moveably supported within the housing, whereas riser pipe and nozzle are firmly and immovably, or stationary, attached to the housing and/or nozzle.

In that it relies on the use of a particularly advantageous inhalation devices, the method provides an improvement to the methods known from the art, wherein the component corresponding to the riser pipe, namely the hollow piston, is moveable with respect to housing and nozzle, and the pumping chamber is stationary.

According to further preferred embodiments, the described method is carried out by means of an inhalation device that provides one, several, or all of the aforementioned features, such as: a reservoir, being firmly attached to the pumping chamber or being firmly attached to the housing, and optionally being provided by a collapsible bag; a check valve, optionally operable by using a threshold pressure difference; a means for the storage of potential energy such as e.g. a spring; a locking device; and an outlet valve, being e.g. arranged inside the riser pipe or between riser pipe and nozzle. For explanations regarding the respective operation of said features, reference is made to the respective sections of the description.

As being shown, the invention solves the drawbacks of the known art. It also allows for an ejection of media of higher viscosities in a short time, and with high reproducibility.

### DESCRIPTION OF FIGURES

FIG. 1 shows schematically a preferred embodiment of the nebulizer for medically active liquids prior to its first use.

FIG. 2 shows a device similar to the one of FIG. 1, but without optional outlet valve.

FIG. 3 shows the embodiment of FIG. 1 before initially filling the pumping chamber.

FIG. 4 shows the situation during the first activation.

FIG. 5 shows the situation at the end of the first activation.

FIG. 6 shows the situation after re-filling the pumping chamber.

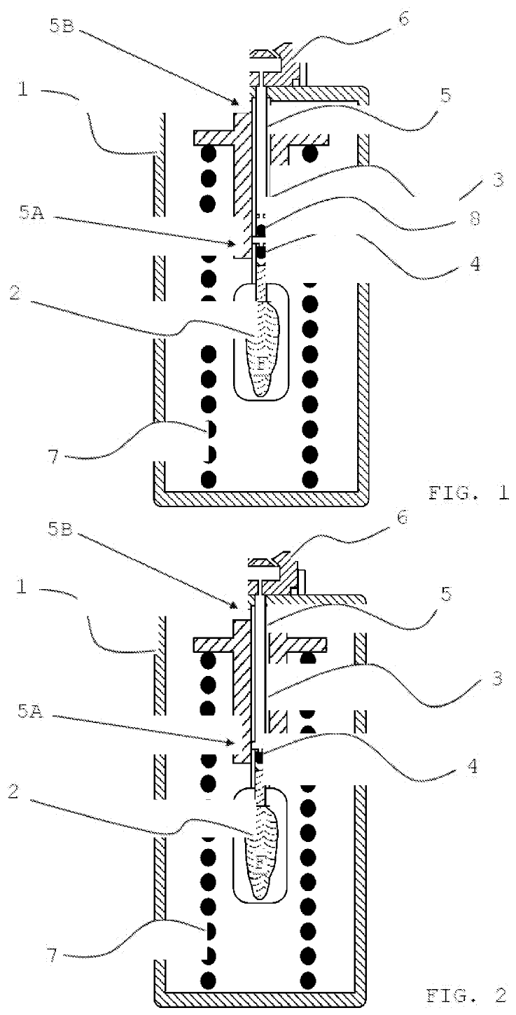

In FIG. 1, a preferred embodiment of the inhalation device for medically active liquids is depicted schematically and not-to-scale. FIG. 1 shows the situation prior to first use.

The inhalation device comprises a housing 1, which is preferably shaped and dimensioned such that it can be held with one hand and can be operated by one finger, e.g. the thumb (not shown). A reservoir 2 for storage of a medically active liquid F is located inside the housing 1. The depicted reservoir 2 is designed to be collapsible; that means that during proceeding emptying, the elastic or at least limp walls buckle, so that the underpressure which is necessary for extraction of a certain amount of liquid is not, or almost not, increased. A similar effect can be achieved when a rigid container has a moveable bottom by means of which the interior volume of the reservoir can also be successively be reduced (not shown).

Further, the inhalation device comprises a pumping device with a pumping chamber 3 within the housing 1 for generation of the desired pressure which is necessary for emitting liquid F and nebulizing the same. The pumping device can also comprise additional, not depicted components (push button, locking device, etc.).

Pumping chamber 3 is fluidically connected with reservoir 2 by means of an inlet check valve 4. Check valve 4 serves for allowing inflow of liquid F into the pumping chamber 3, and blocks a back flow of liquid F into reservoir 2 upon release of the not-depicted locking mechanism.

As a means for the storage of potential energy 7, a spring is provided which is coupled with one (upwards directed) end to the pumping chamber 3 and which is supported at housing 1 (lower part of the figure).

The inhalation device further comprises a riser pipe 5 with at least one reservoir-facing, interior end 5A which can be received in said pumping chamber 3. In other words, riser pipe 5 can at least partially be pushed into pumping chamber 3, resulting in a decrease of the interior volume of pumping chamber 3. The term “interior volume” describes that volume which extends from the reservoir-facing inlet of the pumping chamber 3 to the place where the interior end 5A of the riser pipe 5 is located. In the depicted situation, riser pipe 5 is almost entirely contained in the pumping chamber 3. As a result, the interior volume, situated between check valve 4 and the interior end 5A of riser pipe 5, is at a minimum.

Preferably, in the section which serves for the reception of the riser pipe, pumping chamber 3 has section with an circular inner cross section that corresponds to the (then also) circular outside cross section of the according riser pipe section. Of course, other cross section shapes are possible as well.

According to the depicted embodiment, check valve 4 is arranged between reservoir 2 and inlet of pumping chamber 3.

Finally, the inhalation device comprises a nozzle 6 which is connected liquid-tight to an exterior end 5B of riser pipe 5. Nozzle 6 can be any known nozzle which is suitable for nebulizing/atomizing liquid. The nozzle 6 which is depicted as an example uses the principle of nebulization by means of two colliding liquid jets. Preferably, the cross sections of the liquid-containing channels are relatively small, and typically, in the region of microns.

Also depicted is an optional outlet valve 8 inside riser pipe 5 for avoiding back flow of liquid or air into the exterior end 5B of the same from the outside. Outlet valve 8 is arranged in the interior end 5A of riser pipe 5. Liquid F can pass outlet valve 8 in direction of nozzle 6, but outlet valve 8 blocks any undesired back flow in the opposite direction.

As can be seen in FIG. 1, riser pipe 5 is designed immobile and firmly attached to housing 1, indicated by the connection in the region of exterior end 5B with housing 1. Riser pipe 5 is also firmly attached to nozzle 6, which in turn is attached to housing 1 as well. On contrary, pumping chamber 3 is designed to be moveable with respect to housing 1 and nozzle 6. The benefits of this design have already been explained; reference is made to the respective sections above.

Referring to FIG. 2, a device similar to the one of FIG. 1 is depicted. However, the embodiment shown in FIG. 2 lacks the (optional) outlet valve 8. All other substantial components are present, and also the function is comparable.

FIG. 3 shows the embodiment of FIG. 1 just before initially filling the pumping chamber 3. Pumping chamber 3 is pulled down, loading the means for the storage of potential energy 7. Outlet valve 8 is closed due to underpressure inside pumping chamber 3, and check valve 4 is open to reservoir 2. Increasingly collapsing walls of reservoir 2 allow its inside pressure remain nearly constant, while pressure inside pumping chamber 3 drops because of the upwards motion pulling pumping chamber 3 off riser pipe 5, increasing the interior volume of pumping chamber 3.

As a result, interior volume of pumping chamber 3 fills with liquid F from the reservoir.

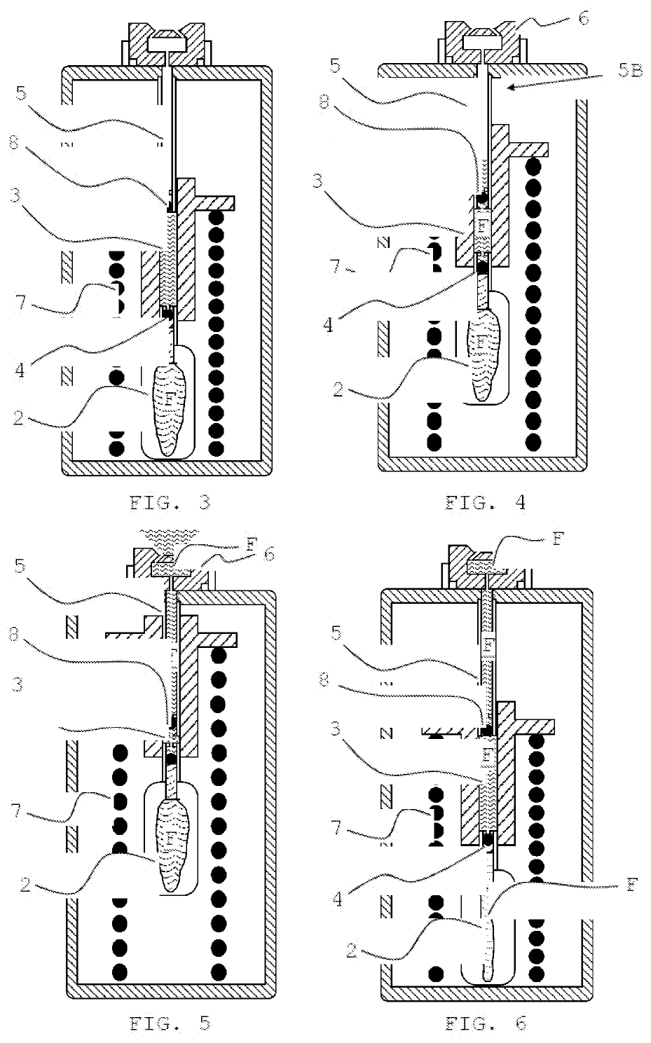

In FIG. 4, the situation during the first activation of the inhalation device is shown. Means for the storage of potential energy 7 has been released from the loaded position as shown in FIG. 3. It pushes the pumping device comprising pumping chamber 3 onto riser pipe 5, the interior end 5A of which coming closer to check valve 4 now being closed. As a result, the pressure inside pumping chamber 3 rises and keeps valve 4 being closed, but opens outlet valve 8. Liquid F rises inside riser pipe 5 towards its exterior end 5B and nozzle 6.

FIG. 5 shows the situation at the end of the first activation. Means for the storage of potential energy 7 is in its most relaxed end position (spring fully extended). Also, pumping chamber 3 has been pushed almost entirely onto riser pipe 5 such that the interior volume of pumping chamber 3 reaches its minimum. Most of liquid F previously contained inside pumping chamber 3 has passed outlet valve 8 into riser pipe 5. Liquid F already contained within riser pipe 5 has been pushed towards, and though, nozzle 6, where the desired nebulization takes place, producing a spray.

In FIG. 6, the situation after re-filling the pumping chamber 3 is depicted. Pumping chamber 3 has again been pulled off interior end 5A of riser pipe 5, increasing the interior volume of pumping chamber 3. Means for the storage of potential energy 7 has been loaded (spring compressed). During movement of pumping chamber 3 away from riser pipe 5, an underpressure develops in the interior volume, closing outlet valve 8 and opening check valve 4. As a result, new liquid F is drawn from reservoir 2 into pumping chamber 3. The inhalation device's pumping chamber 3 is filled again and ready for the next ejection of liquid F by releasing the spring.

### LIST OF REFERENCES

1 housing
2 reservoir
3 pumping chamber
4 check valve
5 riser pipe
5A interior end
5B exterior end
6 nozzle
7 means for the storage of potential energy
8 outlet valve
F liquid

1. An inhalation device for delivering a nebulized medically active aerosol for inhalation therapy, the inhalation device comprising:
a housing configured to receive a fluid reservoir therein;
an impingement-type nozzle fixed to the housing;
an outlet valve configured to prevent backflow; and
a pumping unit located in the housing, the pumping unit comprising:
a riser pipe that is immovably fixed to the housing and in fluid communication with the impingement-type nozzle; and
a hollow cylinder with the riser pipe at least partially inserted into an interior of the hollow cylinder, the hollow cylinder longitudinally translatable along an outer circumference of the riser pipe and configured to be in fluid communication with the fluid reservoir,
an internal space of the riser pipe and hollow cylinder defining a variable volume of the pumping unit, the variable volume of the pumping unit variably based on a longitudinally translational position of the hollow cylinder along the outer circumference of the riser pipe; and
a spring mechanically coupled to the hollow cylinder, the spring configured to vary the variable volume of the pumping unit by changing the longitudinally translational position of the hollow cylinder along the outer circumference of the riser pipe.

2. The inhalation device according to claim 1, wherein
the outlet valve is located in the riser pipe.

3. The inhalation device according to claim 1, wherein
the outlet valve is located at an end of the riser pipe.

4. The inhalation device according to claim 1, wherein
the outlet valve is located between the riser pipe and the impingement-type nozzle.

5. The inhalation device according to claim 1, wherein
the outlet valve is configured to open when a pressure difference between an upstream side of the outlet valve and a downstream side of the outlet valve is greater than a predefined threshold value.

6. The inhalation device according to claim 1, wherein,
the outlet valve is selected from a group consisting of a ball valve, a duck bill valve, and a flap valve.

7. An inhalation device for delivering a nebulized medically active aerosol for inhalation therapy, the inhalation device comprising:
a housing configured to receive a fluid reservoir therein;
an impingement-type nozzle fixed to the housing;
an outlet valve configured to prevent liquid and/or gas backflow; and
a pumping unit located in the housing, the pumping unit in fluid communication with the impingement-type nozzle and the fluid reservoir, the pumping unit comprising:
a riser pipe; and
a hollow cylinder with the riser pipe at least partially inserted into an interior of the hollow cylinder, with the hollow cylinder and the riser pipe longitudinally translatable relative to one another,
an internal space of the riser pipe and hollow cylinder defining a variable volume of the pumping unit, the variable volume of the pumping unit variably based on a longitudinally translational position of the hollow cylinder and the riser pipe relative to one another; and
a spring mechanically coupled to the pumping unit, the spring configured to vary the variable volume of the pumping unit by changing the longitudinally translational position of the hollow cylinder and the riser pipe relative to one another.

8. The inhalation device according to claim 7, wherein
the outlet valve is located between the pumping unit and the impingement-type nozzle.

9. The inhalation device according to claim 8, wherein
the outlet valve is located at within an end of the riser pipe.

10. The inhalation device according to claim 9, wherein
the outlet valve is located at a downstream end of the riser pipe.

11. The inhalation device according to claim 7, wherein
the outlet valve is located between the riser pipe and the impingement-type nozzle.

12. The inhalation device according to claim 8 further comprising:
a connector located between the pumping unit and the impingement-type nozzle, the connector comprising the outlet valve.

13. The inhalation device according to claim 8, wherein
a diameter of the outlet valve is larger than an internal diameter of the riser pipe.

14. The inhalation device according to claim 12, wherein
a diameter of the outlet valve is larger than an internal diameter of the riser pipe.

15. The inhalation device according to claim 7, wherein
the outlet valve is configured to open when a pressure difference between an upstream side of the outlet valve and a downstream side of the outlet valve is greater than a predefined threshold value.

16. The inhalation device according to claim 15, wherein
the predefined threshold value is in a range from 1 to 20 mbar.

17. The inhalation device according to claim 7, wherein,
the outlet valve is a ball valve.

18. The inhalation device according to claim 7, wherein,
the outlet valve is a duck bill valve.

19. The inhalation device according to claim 7, wherein,
the outlet valve is a flap valve.

20. The inhalation device according to claim 7, further comprising:
an inlet check valve configured to prevent backflow that is located between the pumping unit and the fluid reservoir.
